(19) United States
(12) Patent Application Publication
(10) Pub. No.: US 2009/0286832 A1
(43) Pub. Date: Nov. 19, 2009

(54) NARCOTIC EMULSION FORMULATIONS FOR TREATMENT OF SURGICAL PAIN

(76) Inventors: Kiichiro Nabeta, Setagaya-ku (JP); Toru Hibi, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/424,382

(22) Filed: Apr. 15, 2009

Related U.S. Application Data
(60) Provisional application No. 61/053,571, filed on May 15, 2008.

(51) Int. Cl. A61K 31/4468; A61P 29/00

(52) U.S. Cl. 514/329

(57) ABSTRACT

Methods and compositions of treating a subject for post-surgical pain are provided. In the subject methods, a subject is treated for post-surgical pain by administering to the subject an effective amount of a narcotic emulsion, e.g., fentanyl emulsion, formulation. In certain embodiments, the emulsion formulations include a narcotic active agent, oil, water and an surfactant. Also provided are methods of making the subject emulsion formulations as well as kits that include the emulsion formulations.

### CROSS-REFERENCE TO RELATED APPLICATIONS

Pursuant to 35 U.S.C. §119 (e), this application claims priority to the filing date of U.S. Provisional Patent Application Ser. No. 61/053,571 filed May 15, 2008; the disclosure of which is herein incorporated by reference.

### INTRODUCTION

Pain can be defined as an unpleasant sensory and emotional experience associated with actual or potential tissue damage. It is a complex process influenced by both physiological and psychological factors. Pain is typically subjective and many health care professionals are not trained to effectively assess or treat pain.

Over twenty million patients have surgical procedures each year. Post-surgical pain (interchangeably termed, post-incisional pain), or pain that occurs after surgery or traumatic injury, is a serious and often intractable medical problem.

Pain is usually localized within the vicinity of the surgical site. Post-surgical pain can have two clinically important aspects, namely resting pain, or pain that occurs when the patient is not moving and mechanical pain which is exacerbated by movement (coughing/sneezing, getting out of bed, physiotherapy, etc.). The major problem with post-surgical pain management for major surgery is that the drugs currently used have a variety of prominent side effects that delay recovery, prolong hospitalization and subject certain vulnerable patient groups to the risk of serious complications.

The three major classes of pharmaceutical drugs used to treat post-surgical pain are the opioid analgesics, local anesthetics, and the non-steroidal anti-inflammatory drugs (NSAID). Two of these classes of drugs, the opioid analgesics and NSAIDs, are typically administered systemically while the local anesthetics (e.g. channel blockers) are administered non-systemically during surgery.

The systemic administration of drugs to relieve pain after surgery is frequently inadequate. For example, systemic administration of opioids after surgery may cause nausea, the inhibition of bowel function, urinary retention, inhibition of pulmonary function, cardiovascular effects, and sedation.

One opioid analgesic that has been employed for the treatment of post-surgical pain is fentanyl. Fentanyl is the generic name for the compound N-(-phenethyl-4 piperidyl) propionanilide, a useful injectable analgesic. See U.S. Pat. No. 3,164,600. Fentanyl is an opioid agonist and shares many of the pharmacodynamic effects of opioids such as morphine and meperidine. However, compared to these opioids, fentanyl exhibits little hypnotic activity, rarely induces histamine release, and respiratory depression is more short-lived. Fentanyl is commercially available for intravenous, intrabuccal (lozenge-transmucosal) and transdermal administration.

Various injectable fentanyl formulations have been developed to date. One such formulation is a fentanyl citrate composition sold in the United States under the brand name SUBLIMAZE™ that includes fentanyl citrate, USP water for injection, and sufficient sodium hydroxide to raise the pH to 6.5. A different fentanyl citrate composition has been sold in Europe under the brand name FENTANEST™ which consists only of fentanyl and USP water for injection without any deliberate pH adjustment.

Despite the utility of injectable fentanyl formulations, certain drawbacks of such formulations do exist. For example, injectable fentanyl formulations can cause unwanted central nervous system mediated side effects, such as respiratory depression, sedation and dizziness.

As such, there is a need for the development of an injection formulation that is as effective as current injection formulations and yet has a reduced incidence of central nervous system mediated side effects.

### SUMMARY

Methods and compositions of treating a subject for post-surgical pain are provided. In the subject methods, a subject is treated for post-surgical pain by administering to the subject an effective amount of a narcotic emulsion, e.g., fentanyl emulsion, formulation. In certain embodiments, the emulsion formulations include a narcotic active agent, oil, water and a surfactant. Also provided are methods of making the subject emulsion formulations as well as kits that include the emulsion formulations.

### BRIEF DESCRIPTION OF THE DRAWINGS

FIG. 1 shows the effects of Fentanest™ on pain-related scores vs. time in mice.

FIG. 2 shows the effects of fentanyl emulsion A on pain-related scores vs. time in mice.

FIG. 3 shows the inhibition of pain by Fentanest™ and fentanyl emulsion A. The effects are expressed as area under curve (% of Fentanest™).

FIG. 4 shows the duration of Straub's tail reaction induced by Fentanest™ and fentanyl emulsion A. Preparations were administered intravenously in a volume of 0.10 ml. Straub's tail reaction was observed for 1 hour after administration and duration of the response was summed. Results shown are mean ±SEM of animals which showed the response. Figures in the columns indicate the number of animals which showed Straub's tail reaction. Six animals per group were given Fentanest™ or fentanyl emulsion A.

FIG. 5 shows one-direction locomotor activity (e.g. duration of round behavior) induced by Fentanest™. Fentanest™ and vehicle were administered intravenously in a volume of 0.10 ml. Results shown are mean±SEM of six animals. *p<0.05 (Dunnett's multiple comparisons).

FIG. 6 shows one-direction locomotor activity (e.g. duration of round behavior) induced by fentanyl emulsion A. Fentanyl emulsion A and vehicle were administered intravenously in a volume of 0.1(0 ml. Results shown are mean±SEM of six animals. *p<0.05 (Dunnett's multiple comparisons).

FIG. 7 shows a comparison of one-direction locomotor activity (e.g. duration of round behavior) induced by Fentanest™ and fentanyl emulsion A. Results shown are mean±SEM of six animals. *p<0.05 (Dunnett's multiple comparisons).

### DETAILED DESCRIPTION

Methods and compositions of treating a subject for post-surgical pain are provided. In the subject methods, a subject is treated for post-surgical pain by administering to the subject an effective amount of a narcotic emulsion, e.g., fentanyl emulsion, formulation. In certain embodiments, the emulsion formulations include a narcotic active agent, oil, water and a surfactant. Also provided are methods of making the subject emulsion formulations as well as kits that include the emulsion formulations.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In the following sections, the emulsion formulations and methods using the emulsion formulations are described first in greater detail, followed by a review of methods for preparing the formulations and kits that may include the formulations.

### Narcotic Emulsion Formulations

The subject narcotic emulsion formulations include a narcotic active agent. Narcotic active agents of interest are opioid receptor agonists. Opioid receptor agonists include opiates and opioids. “Opiates” and “Opioids” are roughly synonymous terms that generically denote a class of narcotic compounds characterized by having addiction-forming or addiction-sustaining properties similar to morphine or being capable of conversion into a drug having such addiction-forming or addiction-sustaining properties. Specifically, the term “opiates” denotes compounds containing the fundamental morphine or thebaine structure and possessing some affinity to any, or all, of the opioid receptor subtypes. Examples of opiates are heroin, buprenorphine, and naltrexone. An “opioid” is any compound, peptide or otherwise, which, while not containing the fundamental morphine or thebaine structure, possesses some affinity for any, or all, of the opioid receptor subtypes. A non-exclusive list of opiates and opioids includes morphine, heroin, opium, cocaine, fentanyl, ecgonine, thebaine, etc. Commercially-available opiates and opioids (and exemplary trademarked names, where available) include: alfentanil (“Alfenta”), buprenorphine (“Temgesic,” or “Subutex”), carfentanil (“Carfenta”), codeine, dihydrocodeine, diprenorphine, etorphine (“Immobilon”), fentanyl (“Sublimaze” or “Fentanest”), heroin, hydrocodone (“Vicodin”), hydromorphone (“Dilaudid”), LAAM (“Orlaam”), levorphanol (“Levo-Dromoran”), meperidine (“Demerol”), methadone (“Dolophine”), morphine, naloxone (“Narcan”), naltrexone (“Trexan”), beta-hydroxy 3-methylfentanyl, oxycodone (“Percodan”), oxymorphone (“Numorphan”), propoxyphene (“Darvon”), remifentanil (“Ultiva”), sufentanil (“Sufenta”), tilidine (“Valeron”), and tramadol (“Ultram”). The definition includes all opiates and opioids, from any source, including naturally-derived compounds, synthetic compounds, and semi-synthetic compounds. The definition also includes all isomers, stereoisomers, esters, ethers, salts, and salts of such isomers, stereoisomers, esters, and ethers, whenever the existence of such isomers, stereoisomers, esters, and ethers is possible within the specific chemical designation.

As the narcotic formulations described herein are emulsions, the formulations are liquid preparations that are a suspension of small globules of one liquid in a second liquid with which the first liquid will not mix. In certain embodiments, the subject emulsion formulations include a narcotic active agent, oil, water and a surfactant. The narcotic active agent can include an opioid, as described above, where, in some cases, the opioid includes fentanyl, i.e., N-(1-phenethyl-4 piperidyl)propionanilide.

An aspect of the subject emulsion formulations is that the emulsion formulations include an effective amount of a narcotic active agent. By effective amount is meant a dosage sufficient to provide the desired result. For example, if the active agent is an anesthetic, an effective amount is that which provides the desired anesthetic result. As will be apparent to those of skill in the art, the effective amount may vary depending on the particular active agent employed, the particular wound being treated, etc. The amount of narcotic active agent in the subject emulsion formulations may vary, and in certain embodiments ranges from 0.01 to 100mg/ml, such as 0.1 to 50 mg/ml and including 0.1 to 10 mg/ml. In certain embodiments, the emulsion formulations include an effective amount of fentanyl. Fentanyl may be present in the emulsion formulation as the free base or a physiologically acceptable salt thereof, or a hydrate thereof. In certain embodiments, fentanyl is present in the composition at a concentration of 0.05 mg/ml or higher, including 0.1 mg/ml or higher, and in certain embodiments ranges from 0.1 to 10 mg/ml, such as 0.1 to 2 mg/ml, including 0.1 to 1 mg/ml.

In certain embodiments, the emulsions formulations are emulsions of water and oil. As the formulations are emulsions, they are mixtures of two immiscible (e.g. unblendable) fluids, where one fluid (e.g. an oil or water) (the dispersed phase) is dispersed in the other fluid (e.g. the other of the oil or water) (the continuous phase). The water present in the emulsions may be any convenient water, including deinionized water, USP water for injection (WFI), etc.

Also present in certain embodiments of the subject emulsion formulations is an oil. Oils of interest are physiologically acceptable and include, but are not limited to: simple lipids, derived lipids, complex lipids that are derived from natural vegetable oil and fat, animal oil and fat, and mineral oil, or mixtures thereof. In certain embodiments, the oil includes, but is not limited to soybean oil, olive oil, sesame oil, castor oil, corn oil, peanut oil, safflower oil, grape seed oil, eucalyptus oil, medium-chain fatty acid esters, low-chain fatty acid esters, and the like. Animal oils and fat of interest include, but are not limited to, cod-liver oil, seal oil, sardine oil, docosahexiaenoic acid, and eicosapentaenoic acid. Mineral oils of interest include, but are not limited to, liquid paraffins (e.g. oils derived from n-alkanes), naphthenic oils (e.g. oils based on cycloalkanes), and aromatic oils (e.g. oil based on aromatic hydrocarbons). One or a combination of more than one of these types of oils can be used. For example, some embodiments of the subject emulsion formulations include soybean oil, olive oil, sesame oil, or combinations thereof. Other embodiments include soybean oil, olive oil, or combinations thereof. Highly refined oils and fats are employed in certain embodiments. In some instances, the amount of oil in the emulsion formulation ranges from 0.05 to 200 mg/ml, such as 1 to 200 mg/ml and including 10 to 100 mg/ml.

Also present in certain embodiments of the subject emulsion formulations is a surfactant. Surfactants can include any type of surfactant that can be used for pharmaceutical formulations, including but not limited to, phospholipids, refined phospholipids, nonionic surfactants, or mixtures thereof. Refined phospholipids may include phosphatidylinocytol, phosphatidyl ethanolamine, phosphatidylserine, and sphingomyeline with phosphatidylcholine as a main ingredient. For example, refined phospholipids include egg-yolk lecithin and soybean lecithin. Nonionic surfactants of interest include, but are not limited to, polyethylene glycol, polyoxyalkylene copolymer, and sorbitan fatty acid ester. One or a combination of more than one of these surfactants can be used. In certain embodiments, the emulsion formulations include a surfactant, such as a refined phospholipid is employed. In some cases, the emulsion formulations include a refined or hydrogenated phospholipid derived from egg-yolk or soybean with phosphatidylcholine as a main ingredient. The combination ratio of the oil and the surfactant in the subject emulsion formulations is not particularly limited as long as a lipid emulsion can be obtained. As such, the amount of surfactant may vary, ranging in certain embodiments from 0.1 to 50 mg/ml, such as 0.1 to 25 mg/ml, including 1 to 20 mg/ml.

Certain embodiments of the subject emulsion formulations also include one or more emulsification enhancers. Any type of fatty acid that can be used for pharmaceutical formulations can be used as an emulsification enhancer. Of interest are fatty acids that include from 6 to 22 carbons. Either natural or synthetic, and either saturated fatty acids or unsaturated fatty acids can be used, including but not limited to stearic acid, oleic acid, linoleic acid, palmitic acid, linolenic acid, myristic acid, and the like. In certain embodiments, the emulsion formulation includes a refined fatty acid, e.g., oleic acid. The amount of emulsification enhancer included in the emulsion formulation can range from 0.1 to 10 mg/ml, such as from 1 to 5 mg/ml.

Additionally, the emulsion formulations can have a physiologically acceptable pH. In certain embodiments, the pH of the emulsion formulations ranges from 3 to 8, such as from 5 to 7.5, including from 6 to 7. In some instances, the emulsion formulations include a pH adjusting agent. pH adjusting agents of interest include, but are not limited to, sodium hydroxide, hydrochloric acid, phosphoric acid buffer solution, citric acid buffer solution, and the like. For example, the pH of the emulsion formulations can be adjusted to the desired range by adding an appropriate amount of the pH adjusting agent.

Other additives that may be present in the formulation include stabilizing agents, such as but not limited to, glycerin, propylene glycol, polyethylene glycol (e.g., having an average molecular weight of 400 or less), U-glucose, and maltose. Such agents may be included in the subject emulsion formulations in an amount ranging from 0.1 to 50 mg/ml, such as 1 to 25 mg/ml.

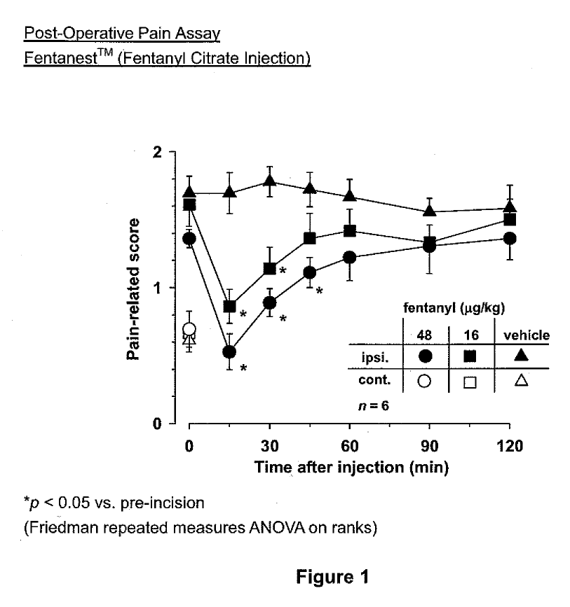

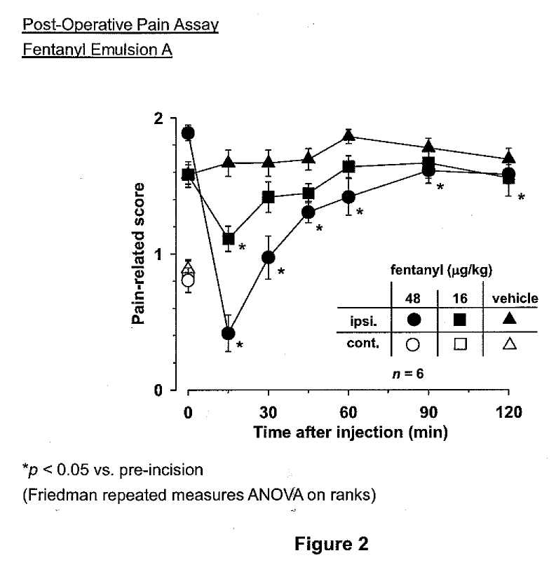

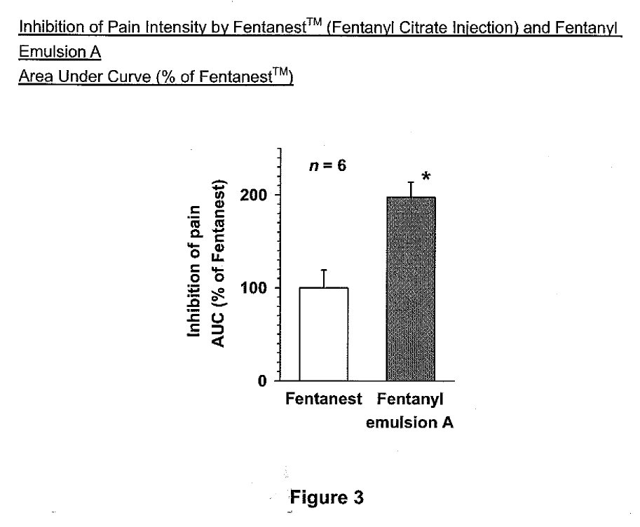

The subject fentanyl containing emulsion formulations can exhibit increased efficacy as compared to Fentanest™ fentanyl citrate injection formulation (0.1 mg/2 ml, available from Sankyo Corporation, Tokyo, Japan). Of interest are emulsion formulations, such as fentanyl emulsion A, which exhibit greater efficacy than Fentanest™ fentanyl citrate injection formulation, as illustrated in the Experimental section below. Efficacy can be measured by comparing the suppression of pain intensity (e.g. pain-related score) between Fentanest™ fentanyl citrate injection formulation and the emulsion described herein (e.g., fentanyl emulsion A) at various time points after administration. In certain embodiments, the emulsion formulations described herein at least exhibit comparable efficacy as compared to Fentanest™ fentanyl citrate injection formulation. For example, the emulsion formulations can exhibit an efficacy 5% or greater, such as 10% or greater, including 15% or greater than Fentanest™ fentanyl citrate injection formulation. In certain embodiments, Fentanest™ fentanyl citrate injection formulation and fentanyl emulsion A suppress pain intensity (e.g. pain-related score) by 61% and 78%, respectively, 15 min after administration, and 35% and 49%, respectively, 30 min after administration, as illustrated in FIGS. 1 and 2. In addition, in some cases, the reduction of pain intensity (e.g. pain-related score) by fentanyl emulsion A, expressed as area under a curve, is 50% or greater, such as 75% or greater, including 100% or greater than the area under a curve for Fentanest™ fentanyl citrate injection formulation. In some cases, the subject emulsion formulations can reduce pain intensity expressed as area under a curve by twice as much as that of Fentanest™ fentanyl citrate injection formulation, as illustrated in FIG. 3.

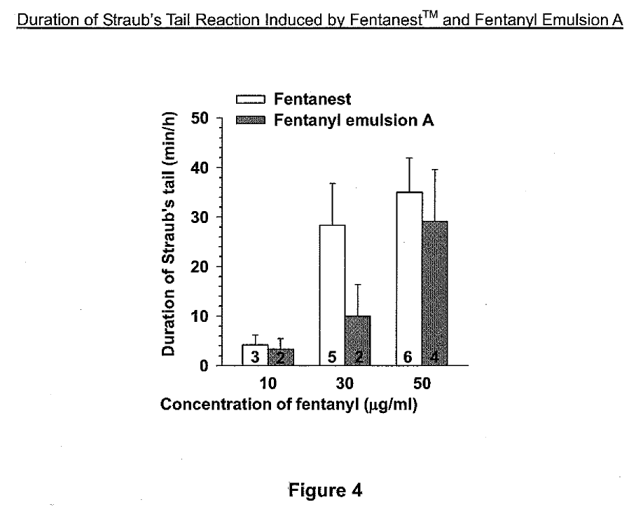

In certain embodiments, the emulsion formulations described herein exhibit reduced central nervous system mediated side effects as compared to Fentanest™ fentanyl citrate injection formulation. A reduction in central nervous system mediated side effects can be observed by comparison of the incidence of Straub's tail reaction between the emulsion formulations described herein and the Fentanest™ fentanyl citrate injection formulation. Straub's tail reaction in mice is an S-shaped dorsiflexion of the mouse tail, which can be used as a sensitive and specific bioassay for opioids. Straub's tail reaction is mediated by the action of opioids on the sacro-coccygeal dorsalis muscles at the level of the lumbosacral spinal cord. In certain instances, the emulsion formulations described herein exhibit a reduced incidence of Straub's tail reaction as compared to a Fentanest™ fentanyl citrate injection formulation. For example, following administration of a 30 μg/ml dose of fentanyl emulsion A, as illustrated in Table 5 below, the incidence of Straub's tail reaction in mice was 50% or less, such as 33% or less as compared to Fentanest™ fentanyl citrate injection formulation, as determined using the assay protocol reported in the Experimental section below. In certain instances, the duration of Straub's tail reaction for fentanyl emulsion A was 80% or less, such as 75% or less, including 36% or less than that of Fentanest™ fentanyl citrate injection formulation, as illustrated in FIG. 4.

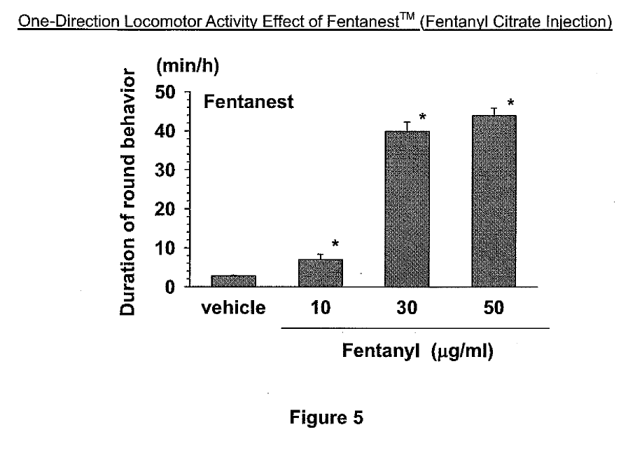

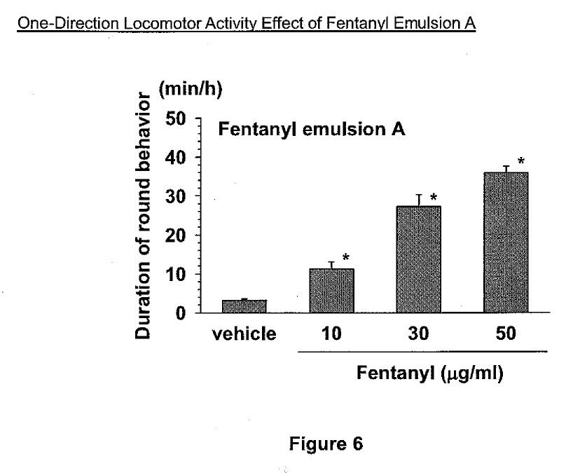

Aspects of the emulsion formulations disclosed herein also include emulsion formulations that exhibit reduced brain mediated side effects (such as an increase in one-direction locomotor activity, e.g., round behavior), as compared to Fentanest™ fentanyl citrate injection formulation. For example, the duration of round behavior following administration of a 30 μg/ml dose of Fentanest™ fentanyl citrate injection formulation or fentanyl emulsion A was 40 and 27 min, respectively, as illustrated in FIGS. 5 and 6, as determined using the assay protocol reported in the Experimental section below. In certain embodiments, the duration of round behavior is 40 min or less, such as 35 min or less, including 30 min or less following administration of the subject emulsion formulation.

Additional aspects of the subject emulsion formulations include emulsion formulations that are storage-stable. By storage-stable is meant that the compositions may be stored for extended periods of time without significant phase separation and/or significant reduction in activity of the active agent. In certain embodiments, the subject compositions are stable for 6 months or longer, such as 1 year or longer, including 3 years or longer, etc., when maintained at 25° C. By the phrase “without significant reduction in the activity of the active agent” is meant that at the end of the storage period, there is a reduction of 10% or less, such as 5% or less, including 3% or less in the activity of the active agent compared to the activity of the active agent at the beginning of the storage period.

Methods of Treating Post-Surgical Pain with Narcotic Emulsion Formulations

As summarized above, provided are methods of treating a subject for post-surgical pain. “Post-surgical pain” (interchangeably termed “post-incisional” or “post-traumatic pain”) refers to pain arising or resulting from an external trauma such as a cut, puncture, incision, tear, or wound into tissue of an individual (including those that arise from all surgical procedures, whether invasive or non-invasive). As used herein, “post-surgical pain” does not include pain that occurs without an external physical trauma. In some embodiments, post-surgical pain is internal or external pain, and the wound, cut, trauma, tear or incision may occur accidentally (as with a traumatic wound) or deliberately (as with a surgical incision). As used herein, “pain” includes nociception and the sensation of pain, and pain can be assessed objectively and subjectively, using pain scores and other methods, e.g., with protocols well-known in the art. Post-surgical pain, as used herein, includes allodynia (i.e., pain due to a stimulus that does not normally provoke pain) and hyperalgesia (i.e., increased response to a stimulus that is normally painful), which can in turn, be thermal or mechanical (tactile) in nature. In some embodiments, the pain is characterized by thermal sensitivity, mechanical sensitivity and/or resting pain (e.g. persistent pain in the absence of external stimuli). In some embodiments, the post-surgical pain includes mechanically-induced pain or resting pain. In other embodiments, the post-surgical pain includes resting pain. The pain can be primary (e.g., resulting directly from the pain-causing event) or secondary pain (e.g., pain associated with, but not directly resulting, from the pain-causing event).

Accordingly, in one aspect, provided are methods of treating a subject for post-surgical pain that include administering an effective amount of an emulsion formulation of a narcotic active agent. In some embodiments, the post-surgical pain includes one or more of; allodynia, hyperalgesia, thermally induced pain, mechanically induced pain, or resting pain. For instance, post-surgical pain can include mechanically induced pain and/or resting pain. In some cases, the post-surgical pain includes resting pain. By “treating” or “treatment” is meant at least a suppression or an amelioration of the symptoms associated with the condition afflicting the subject, where suppression and amelioration are used in a broad sense to refer to at least a reduction in the magnitude of a parameter, e.g., symptom, associated with the condition being treated, such as pain. As such, treatment also includes situations where the condition is completely inhibited, e.g., prevented from happening, or stopped, e.g., terminated, such that the subject no longer experiences the condition. As such, treatment includes both preventing and managing a condition. In certain embodiments, allodynia is suppressed, ameliorated and/or prevented, and in some embodiments, hyperalgesia is suppressed, ameliorated and/or prevented. In some instances, the pain is chronic pain. In-other cases, the pain is at, proximal and/or near to one or more site(s) of external trauma, wound or incision.

Additional aspects of the subject methods include methods of ameliorating and/or preventing the development or progression of post-surgical pain by administering the subject emulsion formulations. In certain embodiments, the emulsion formulations can be administered prior to an activity likely to result in external trauma, wound or incision, such as surgery. For example, the emulsion formulation can be administered 30 minutes, 1 hour, 2 hours, 5 hours, 10 hours, 15 hours, 24 hours or even more, such as 1 day, several days, or even a week, two weeks, three weeks, or more prior to the activity likely to result in external trauma, wound or incision, such as prior to surgery. In other embodiments, the emulsion formulation can be administered during and/or after surgery or activity that resulted in external trauma, wound or incision. In some instances, the emulsion formulation is administered 1 hour, 2 hours, 3 hours, 4 hours, 6 hours, 8 hours, 12 hours, 24 hours, 30 hours, 36 hours, or more, after surgery, or activity that resulted in external trauma, wound or incision.

In practicing the methods, the emulsion formulations disclosed herein can be parenterally administered to a subject. By “parenteral administration” is meant administration by a protocol that delivers a quantity of the emulsion formulation to the subject, e.g., a patient suffering from post-surgical pain, by a route other than the digestive tract. Examples of parenteral administration include, but are not limited to, intramuscular injection, intravenous injection, transdermal absorption, inhalation, and the like. In certain embodiments, parenteral administration is by injection using an injection delivery device. The amount of emulsion formulation that is administered to the subject may vary depending on a number of factors, such as patient specifics, prior opiate treatment, nature of pain, etc. In certain embodiments, the dosage of active agent that is administered per dosing event ranges from 10 to 250 μg/dose, such as 10 to 150 μg/dose, including 25 to 100 μg/dose. Dosing guidelines for emulsion formulations already developed and followed by those of skill in the art may be employed with the subject emulsion formulations.

Further aspects of the subject methods include methods for increasing pain threshold. As used herein, “increasing pain threshold” refers to a reduction, diminishment and/or minimization of pain associated with surgery, incision, trauma or wound (including reduced, diminished, and/or minimized subjective perception of pain). In yet another aspect, the subject methods provide for enhancing recovery from surgery, as well as enhancing recovery from wound, traumatic injury, and/or incision.

It is appreciated that although reference is generally made herein to treating or preventing post-surgical pain, the emulsion formulations can be administered before an activity with an increased risk of external trauma (such as an impact), injury, or wound. As is understood by one skilled in the art, an activity with increased risk of external trauma, injury or wound encompasses dangerous vocations, combat, and/or sporting activities.

In certain embodiments, the subject methods include a diagnostic step. Individuals may be diagnosed as being in need of the subject methods using any convenient protocol. In addition, individuals may be known to be in need of the subject methods, e.g., they are suffering from a target disease condition or have been determined to be at risk for suffering from a target disease condition, prior to practicing the subject methods.

Diagnosis or assessment of pain is well-established in the art. Assessment may be performed based on objective measure, such as observation of behavior such as reaction to stimuli, facial expressions and the like. Assessment may also be based on subjective measures, such as patient characterization of pain using various pain scales. See, e.g., Katz et al, Surg. Clin. North Am. (1999) 79 (2):231-52; Caraceni et al. J. Pain Symptom Manage (2002) 23(3):239-55.

Pain relief may also be characterized by time course of relief. Accordingly, in some embodiments, pain relief is subjectively or objectively observed after 1, 2, or a few hours (and in some embodiments, peaks at about 12-18 hours). In other embodiments, pain relief is subjectively or objectively observed at 24, 36, 48, 60, 72 or more hours following surgery (or activity associated with wound or trauma).

### Preparation Methods

The subject emulsion formulations can be prepared using any convenient emulsification protocol. In certain embodiments, the preparation methods include mixing an active agent, water and oil, and emulsifying the mixture. For example, an injection solvent, e.g., WFI, can be added to a smooth mixture of a suitable oil. Initially, the mixture can be roughly emulsified. For example, for rough emulsification, Homomixer (Mizuho Industrial Co., Ltd.) or High Flex Disperser (SMT) can be used. After the mixture is roughly emulsified, the mixture can be finely emulsified, e.g., by using a high pressure emulsification machine. For fine emulsification, a high pressure homogenizer such as Gaulin Homogenizer (APV-SMT) and Microfluidizer (Microfluidics, Newton, Mass.) can be used. In addition, for fine emulsification, the emulsion formulation may be treated by the emulsification machine more than once, such as 2 to 50 times, for example 5 to 20 times, at a pressure ranging from 500 to 850 kg/cm². The preparation methods can be carried out at room temperature or at a temperature lower than room temperature. In certain embodiments, the preparation methods include flushing the emulsification machine with nitrogen gas.

### Utility

The subject emulsion formulations and methods find use in a variety of applications, including preventing or treating post-surgical pain. Accordingly, the subject emulsion formulations and methods are useful for treating, delaying development of and/or preventing post-surgical pain in subjects, including all mammals, both human and non-human, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans. Moreover, the subject emulsion formulations and methods are useful in individuals having an incisional wound to tissue whether a cut, puncture or tear, whether internal or external. Such an incisional wound may occur accidentally as with traumatic wound or deliberately as with surgery.

### Kits

Also provided are kits that find use in practicing the subject methods, as described above. For example, kits for practicing the subject methods may include a quantity of the emulsion formulation, present in unit dosages, e.g., ampoules, or a multi-dosage format. As such, in certain embodiments, the kits may include one or more unit dosages (e.g., ampoules) of the emulsion formulation. The term “unit dosage”, as used herein, refers to physically discrete units suitable as unitary dosages for human and animal subjects, each unit containing a predetermined quantity of the subject emulsion formulation calculated in an amount sufficient to produce the desired effect. The amount of the unit dosage of the subject emulsion formulation depends on various factors, such as the particular active agent employed, the effect to be achieved, and the pharmacodynamics associated with the active agent in the subject. In yet other embodiments, the kits may include a single multi dosage amount of the emulsion formulation.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., one or more pieces of paper on which the information is printed, in the packaging of the kit, in a package insert, etc. The instructions may be present on a computer readable medium, e.g., diskette, CD, DVD, etc., on which the information has been recorded. The instructions may be present on a website, which may be used via the internet to access the information at a removed site. Other convenient means are possible and may be included in the kits.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Experimental

### I. Fentanyl Emulsion Formulation & Preparation

### A. Formulation

(Emulsion, 250 ml)

TABLE 1
Ingredients for the oil phase

| | Emulsion A |
|---|---|
| Soybean Oil | 25 g |
| Refined Egg-Yolk | 4.5 g |
| Phospholipid | |
| Oleic Acid | 0.6 g |

TABLE 2
Ingredients for the aqueous phase (Glycerin solution)

| | Emulsion A | |
|---|---|---|
| Glycerin | 5.52 | g |
| Purified Water | 200 | ml |

### B. Procedure for the Preparation of Fentanyl Emulsion A

Ingredients for the oil phase were added to a beaker and heated to 60° C. while stirring at 7,000 rpm to dissolve the ingredients.

25 mg of Fentanyl was added and stirred. (The process took approximately 10 minutes.)

50 ml of the glycerin solution was added in drops into the mixture and stirred at 10,000 rpm for 10 minutes.

The solution was transferred into a separable flask. The remaining glycerin solution was added while stirring at 12,000 rpm in an emulsion machine for 30 minutes.

Purified water was added to bring the total volume of the emulsion to 250 ml.

The emulsion was emulsified 20 times while cooling the mixture at a pressure of 650 Bar with a high pressure emulsion machine, LAB-1000 (APV, Denmark).

No adjustment was needed if the pH of the emulsion was between 6 and 7. If not, the pH was adjusted with hydrochloric acid solution or sodium hydroxide solution. No adjustment was made in this experiment as the pH was between 6.3 and 6.7.

After high-pressure emulsification, the emulsion was filtered (pore size of 0.4 μm) and the emulsion was added into ampoules while adding N₂ gas.

The ampoules were sterilized by autoclave sterilization (121° C. for 10 minutes).

After sterilization, the ampoules were cooled and stored.

The average particle size of the sterilized samples was measured using a Zetasizer 3000HS (Malvern Instruments, Worcestershire, UK) using a photo-correlation method.

### II. Post-Surgical Pain Assay of Fentanyl Formulation

The analgesic effects of Fentanest™ and fentanyl emulsion A for post-surgical pain in mice were examined.

### A. Materials and Methods

### Animals

Male C57BL/6Cr mice were used.

Test preparations
1) Fentanest injection (fentanyl citrate, 0.1 mg/2 ml fentanyl)
2) Fentanyl emulsion A (0.1 mg/ml fentanyl; average particle size=181 nm)

### Administration

Each formulation was prepared with appropriate solvent. Each formulation was injected intravenously in an amount of 0.05 ml per 10 g body weight.

### Preparation of Post-Operative Pain Model

An incision was made approximately 1 cm from the heel toward the tiptoe under pentobarbital (50 mg/kg, i.p.) anesthesia. Another 1 cm incision was made in the skin, membrane and muscle with a stainless steel scalpel while avoiding the blood vessels and nerves under the skin. The two incision areas were sutured twice with a No. 7 suture and the wound was disinfected with Isodine (Meiji Seika Kaisha Ltd., Tokyo, Japan).

### Post-Operative Pain Assay

Reaction to a mechanical stimuli to the hind legs was used as an indicator of post-operative pain. Post-operative pain was measured in both hind legs (i.e., ipsi-lateral and contra-lateral to the wound area). A Von Frey filament (North Coast Medical, San Jose, Calif., USA) with the strength of 1.6 mN was used for measurements. After applying the Von Frey filament to the bottom of the foot of the hind leg, the reaction was categorized into three stages (0=no reaction; 1=raising the hind leg; 2=shaking the hind leg and licking the hind leg). The stimuli by Von Frey filament were given while avoiding the wound area. The procedure was repeated six times and the average was considered as the pain reaction score.

The post-operative pain assay was carried out in a blind fashion; experimenters knew that all preparations contained fentanyl but they did not know differences in the features of the preparations.

### B. Results

### Summary

A dose-dependent inhibition of post-operative pain that peaked at 15 minutes after administration was observed and the effects were observed for about 60 minutes (FIGS. 1 and 2). A significant inhibition of post-operative pain was observed in both the Fentanest™ injection and the fentanyl emulsion A groups at doses of 16 μg/kg, and 48 μg/kg (FIGS. 1 and 2).

### Comparison of Inhibiting Effects After Administering Fentanvl or Formulations

No difference was observed in the inhibiting effects after administration between the Fentanest™ injection and fentanyl emulsion A (Two-Way RM ANOVA) (Table 4). No significant difference was observed in the area under the curve for 60 minutes after administration (One-Way ANOVA) (FIG. 3).

TABLE 4
Two-Way RM ANOVA

| | P | F |
|---|---|---|
| Treatment (main effect) | 2.141 | 0.127 |
| Treatment x Time | 0.447 | 0.9737 |
| (interaction) | | |

| | p < 0.05 |
|---|---|
| Fentanyl Citrate injection | no |
| vs. Emulsion A | |

Comparing the inhibiting effects between the Fentanest™ injection and the fentanyl emulsion A at 15 minutes after administration, no significant difference was observed (FIGS. 1 and 2). However, compared with the reference standard, substantial inhibiting effects were observed for fentanyl emulsion A formulation (Bonferroni t-test) (FIG. 2).

Comparing the inhibiting effects between the Fentanest™ injection and the fentanyl emulsion A at 30 minutes after administration, no significant difference was observed (FIGS. 1 and 2). However, compared with the reference standard, lasting effects were observed for fentanyl emulsion A formulation (Bonferroni t-test) (FIG. 2).

### III. Assessment of Central Nervous System Mediated Side Effects of Fentanyl Emulaion Formulation

### A. Straub's Tail Reaction

When mice are given opioids, their tails stand and trend rostrally. This reaction is called Straub's tail reaction and may be mediated by the central nervous system, especially spinal cord. Fentanest™ induced Straub's tail reaction in mice tested at doses of 30 and 50 μg/ml (Table 5). In comparison, the incidence of Straub's tail reaction was lower after injection of fentanyl emulsion A at doses of 30 and 50 μg/ml (Table 5). Similar results were observed in the duration of the Straub's tail reaction (FIG. 4).

TABLE 5
Straub's Tail Reaction Induced by Fentanyl Preparations

| Concentration | Mice | Responder (%) | Responder (%) |
|---|---|---|---|
| (μg/ml) | Observed | Fentanest ™ | Emulsion A |
| 0 | 6 | 0 (0) | 0 (0) |
| 10 | 6 | 3 (50) | 2 (33) |
| 30 | 6 | 5 (83) | 2 (33) |
| 50 | 6 | 6 (100) | 4 (80) |

### B. Locomotor Activity Effect

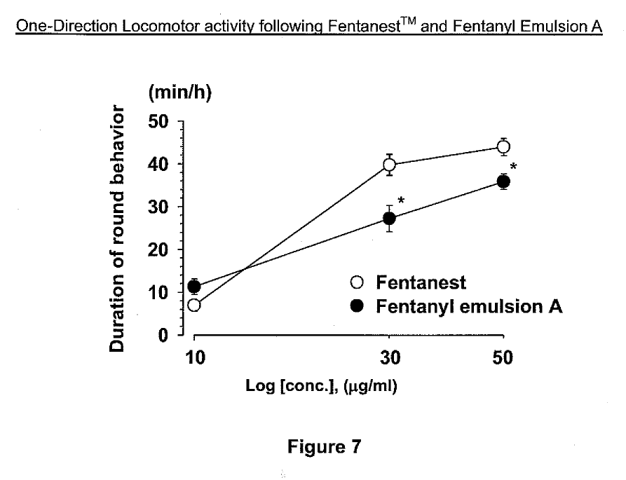

When mice are given high doses of opioids, locomotor activity is increased through the opioid's action on the central nervous system. This increase in locomotor activity can be observed by an increase in one-way locomotor activity (i.e., round behavior). Fentanest™ markedly increased locomotor activity at doses of 30 and 50 μg/ml, but did not have a significant effect at a dose of 10 μg/ml (FIG. 5). The increase in locomotor activity after administration of fentanyl emulsion A was lower than that of Fentanest™ (FIG. 6). The effects of Fentanest™ and fentanyl emulsion A were compared, as shown in FIG. 7.

### C. Discussion

Since Straub's tail reaction and increased locomotor activity may be mainly mediated by the central nervous system, the central nervous system mediated side effects of fentanyl preparations was considered from the results. As compared to Fentanest™, the central nervous system mediated side effects of fentanyl emulsion A were less severe than that of Fentanest™. The incidence of Straub's tail reaction and the duration of round behavior in mice were significantly less with fentanyl emulsion A.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of treating a subject for post-surgical pain, said method comprising:
administering to said subject an amount of a narcotic emulsion formulation effective to treat said subject for post-surgical pain.

2. The method according to claim 1, wherein said narcotic emulsion formulation comprises an opioid.

3. The method according to claim 2, wherein said opioid is fentanyl.

4. The method according to claim 3, wherein said emulsion comprises;
fentanyl;
oil;
water; and
a surfactant.

5. The method according to claim 4, wherein said fentanyl is present in an amount ranging from 0.1 to 10 mg/ml.

6. The method according to claim 5, wherein said oil is present in an amount ranging from 0.05 to 200 mg/ml.

7. The method according to claim 4, wherein said surfactant is chosen from egg-yolk phospholipid or soybean phospholipid.

8. The method according to claim 4, wherein said emulsion formulation further comprises an emulsification enhancer.

9. The method according to claim 8, wherein said emulsification enhancer is oleic acid.

10. The method according to claim 4, wherein said emulsion formulation further comprises glycerin or propylene glycol.

11. The method according to claim 4, wherein said emulsion formulation exhibits comparable efficacy as compared to a Fentanest™ fentanyl citrate injection formulation.

12. The method according to claim 4, wherein said emulsion formulation exhibits reduced central nervous system mediated side effects as compared to a Fentanest™ fentanyl citrate injection formulation.

13. An emulsion formulation comprising:
fentanyl;
oil;
water; and
a surfactant.

14. The emulsion formulation according to claim 13, wherein said fentanyl is present in an amount ranging from 0.1 to 10 mg/ml.

15. The emulsion formulation according to claim 13, wherein said oil is present in an amount ranging from 0.05 to 200 mg/ml.

16. The emulsion formulation according to claim 13, wherein said surfactant is chosen from egg-yolk phospholipid or soybean phospholipid.

17. The emulsion formulation according to claim 13, wherein said emulsion formulation further comprises an emulsification enhancer.

18. The emulsion formulation according to claim 17, wherein said emulsification enhancer is oleic acid.

19. The emulsion formulation according to claim 13, wherein said emulsion formulation further comprises glycerin.

20. The emulsion formulation according to claim 13, wherein said emulsion formulation exhibits comparable efficacy as compared to a Fentanest™ fentanyl citrate injection formulation.

21. The emulsion formulation according to claim 13, wherein said emulsion formulation exhibits reduced central nervous system mediated side effects as compared to a Fentanest™ fentanyl citrate injection formulation.

22. A kit comprising an emulsion formulation comprising:
fentanyl;
oil;
water; and
a surfactant.

23. The kit according to claim 22, wherein said fentanyl is present in an amount ranging from 0.1 to 10 mg/ml.

24. The kit according to claim 22, wherein said oil is present in an amount ranging from 0.05 to 200 mg/ml.

25. The kit according to claim 22, wherein said surfactant is chosen from egg-yolk phospholipid or soybean phospholipid.

26. The kit according to claim 22, wherein said emulsion formulation further comprises an emulsification enhancer.

27. The kit according to claim 26, wherein said emulsification enhancer is oleic acid.

28. The kit according to claim 22, wherein said emulsion formulation further comprises glycerin.

29. The kit according to claim 22, wherein said emulsion formulation exhibits comparable efficacy as compared to a Fentanest™ fentanyl citrate injection formulation.

30. The kit according to claim 22, wherein said emulsion formulation exhibits reduced central nervous system mediated side effects as compared to a Fentanest™ fentanyl citrate injection formulation.
